(19) United States
(12) Patent Application Publication
(10) Pub. No.: US 2022/0340726 A1
(43) Pub. Date: Oct. 27, 2022

(54) GAS PERMEABLE, ULTRATHIN, STRETCHABLE EPIDERMAL ELECTRONIC DEVICES AND RELATED METHODS

(71) Applicant: North Carolina State University, Raleigh, NC (US)

(72) Inventors: Yong Zhu, Apex, NC (US); Weixin Zhou, Nanjing (CN); Shanshan Yao, Coram, NY (US)

(21) Appl. No.: 17/728,182

(22) Filed: Apr. 25, 2022

Related U.S. Application Data
(60) Provisional application No. 63/179,060, filed on Apr. 23, 2021.

(51) Int. Cl. C08J 7/04; H01B 1/02; C08L 75/04; C08K 3/08; H01L 21/02; G06F 3/041; H01B 5/14; A61B 5/00

(52) U.S. Cl. CPC C08J 7/0427; H01B 1/02; C08L 75/04; C08K 3/08; H01L 21/02603; G06F 3/0416; H01B 5/14; A61B 5/6802; B82Y 30/00

(57) ABSTRACT

Presented herein are gas permeable, ultrathin, stretchable epidermal electronic devices and related methods enabled by self-assembled porous substrates and conductive nanostructures. Efficient and scalable breath figure method is employed to introduce the porous skeleton and then silver nanowires (AgNWs) are dip-coated and heat-pressed to offer electric conductivity. The resulting film has a transmittance of 61%, sheet resistance of 7.3 Ω/sq, and water vapor permeability of 23 mg cm⁻² h⁻¹. With AgNWs embedded below the surface of the polymer, the electrode exhibits excellent stability with the presence of sweat and after long-term wear. The present subject matter demonstrates the potential of the electrode for wearable applications—skin-mountable biopotential sensing for healthcare and textile-integrated touch sensing for human-machine interfaces. The electrode can form conformal contact with human skin, leading to low skin-electrode impedance and high-quality biopotential signals. In addition, the textile electrode can be used in a self-capacitance wireless touch sensing system.

### PRIORITY CLAIM

This application claims the benefit of U.S. Provisional Patent Application Ser. No. 63/179,060 filed Apr. 23, 2021, the disclosure of which is incorporated herein by reference in its entirety.

### GOVERNMENT INTEREST

This invention was made with government support under Grant No. CMM11728370 awarded by the National Science Foundation (NSF). The government has certain rights in the invention.

### TECHNICAL FIELD

The subject matter described herein relates to electronic textiles comprising nanowires and the manufacturing of the same. More specifically, the subject matter relates to multi-functional gas-permeable electronic textiles employing nanowires and related methods.

### BACKGROUND

Epidermal electronics have seen a wide range of applications from personal healthcare to human activity monitoring to human-machine interfaces. It is critical that epidermal electronics form conformal contact with the skin in order to improve the quality of sensing signals. Currently, most epidermal devices are built on solid polymeric substrates, such as polydimethylsiloxane (PDMS), polyethylene terephthalate (PET), and polyimide (PI), which lack efficient gas permeability. This can prevent evaporation of sweat and emission of volatile organic components from human skin, leading to skin irritation and reducing the comfort of wearing. Therefore, to achieve long-term wearing, it is imperative to develop gas-permeable materials.

In order to improve gas permeability and achieve conformal contact, a number of architectures have been pursued in recent years, including textiles, meshes, and porous structures. For example, Someya and co-workers recently reported an ultrathin nanomesh electrode with an electrospun poly(vinyl alcohol) (PVA) film, which showed good gas permeability, but the fabrication process was rather complicated. Fan et al. developed an electrospun PVA and silver nanowire (AgNW) conductive composite, which increased the robustness in electrical stability, but the exposed nanowires limited the long-term stability. Another method employed sugar templates to prepare a PDMS sponge and transfer a thin layer of graphene onto the surface of the sponge as the conductive material. Limited by the size of the sugar particles, micropore structures were difficult to prepare with this method. In addition, this method was not feasible to fabricate ultrathin films. It remains challenging to develop gas-permeable and ultrathin materials in a simple and scalable fashion.

### SUMMARY

According to one aspect of the subject matter described herein, a thin film epidermal electronic device includes a polymer film having one or more holes therethrough is provided. The polymer film comprises conductive nanomaterials embedded at or just below a surface of the polymer film. The conductive nanomaterials are connected to form a network of nanomaterials, thereby causing at least a part of the polymer film to act as an electrode. The polymer film is insoluble in water, but soluble in an organic solvent.

According to another aspect of the subject matter described herein, the polymer film comprises thermoplastic polyurethane (TPU), polystyrene-polybutadiene-polystyrene (SBS), or thermoplastic polyolefin (TPO).

According to another aspect of the subject matter described herein, the polymer film has a thickness of between, and including, about 1 μm and 100 μm.

According to another aspect of the subject matter described herein, the conductive nanomaterials comprise silver nanowires (AgNWs), copper nanowires (CuNWs), nickel nanowires (NiNWs), gold nanowires (AuNWs), carbon nanotubes, graphene, or poly(3,4-ethylenedioxythiophene) polystyrene sulfonate (PEDOT:PSS).

According to another aspect of the subject matter described herein, the thin film epidermal electronic device is gas permeable.

According to another aspect of the subject matter described herein, the thin film epidermal electronic device is configured to be attached to human skin, wherein the one or more holes are configured to allow sweat to evaporate from the human skin.

According to another aspect of the subject matter described herein, each of the one or more holes has a diameter of between, and including, about 1 μm and 100 μm.

According to another aspect of the subject matter described herein, at least one of the one or more holes has a diameter of between, and including about 30 μm and 50 μm.

According to another aspect of the subject matter described herein, between, and including, about 30% and 50% of a surface area of the polymer film is covered by the one or more holes.

According to another aspect of the subject matter described herein, the conductive nanomaterials are embedded on both a top surface and a bottom surface of the polymer film. The top surface of the polymer film may be a first surface facing a first direction, where the first direction is an outward facing direction when the thin film epidermal electronic device is worn by a user. The bottom surface may be a second surface facing a second direction that is opposite the first direction.

According to another aspect of the subject matter described herein, conductive nanomaterials are also embedded on an inner surface of each of the one or more holes thereby connecting the conductive nanomaterials on the top surface and the conductive nanomaterials on the bottom surface.

According to another aspect of the subject matter described herein, a garment is provided. The garment includes a thin film epidermal electronic device, wherein the thin film epidermal electronic device includes a polymer film having one or more holes therethrough. The polymer film comprises conductive nanomaterials embedded at or just below a surface of the polymer film. The conductive nanomaterials are connected to form a network of nanomaterials, thereby causing at least a part of the polymer film to act as an electrode. The polymer film is insoluble in water, but soluble in an organic solvent.

According to another aspect of the subject matter described herein, an electrophysiological sensing system is provided. The electrophysiological sensing system includes one or more thin film epidermal electronic devices. The one or more thin film epidermal electronic devices each comprise thin film epidermal electronic device. The thin film epidermal electronic device includes a polymer film having one or more holes therethrough. The polymer film comprises conductive nanomaterials embedded at or just below a surface of the polymer film. The conductive nanomaterials are connected to form a network of nanomaterials, thereby causing at least a part of the polymer film to act as an electrode. The polymer film is insoluble in water, but soluble in an organic solvent.

According to another aspect of the subject matter described herein, a method for making a thin film epidermal electronic device is provided. The method includes creating a polymer layer by adding a solution of a polymer and an organic solvent on a substrate. The polymer is insoluble in water, but soluble in the organic solvent. The method further includes evaporating the organic solvent from the polymer layer. As the organic solvent evaporates, the polymer remains and one or more water droplets form in the polymer layer. The method further includes forming one or more holes in the polymer layer by evaporating the water droplets. The space occupied by a particular water droplet becomes a hole after evaporation of the particular water droplet. The method further includes removing the polymer layer from the substrate. The method further includes embedding conductive nanomaterials in the polymer layer by dip-coating the polymer layer in a solution comprising conductive nanomaterials. The method further includes using a heat-press to adhere the conductive nanomaterials to the polymer layer.

According to another aspect of the subject matter described herein, the polymer comprises thermoplastic polyurethane (TPU) and the organic solvent comprises tetrahydrofuran (THF).

According to another aspect of the subject matter described herein, the method for making the thin film epidermal electronic device includes facilitating an ordered assembly of water droplets in the polymer layer by adding a quantity of polyethylene glycol (PEG) to the solution of TPU and THF, and the PEG evaporates with the THF to leave a thin TPU film behind on the According to another aspect of the subject matter described herein, a ratio of TPU to PEG in the solution is about 10:1 by weight.

According to another aspect of the subject matter described herein, the solution comprises between, and including, about 1% by weight of TPU and 0.1% by weight of PEG and 2% by weight of TPU and 0.2% by weight of PEG.

According to another aspect of the subject matter described herein, the solution comprises about 1.5% by weight of TPU and 0.15% by weight of PEG.

According to another aspect of the subject matter described herein, the polymer layer has a thickness of between, and including, about 1 μm and 100 μm.

According to another aspect of the subject matter described herein, the polymer layer comprises thermoplastic polyurethane (TPU), polystyrene-polybutadiene-polystyrene (SBS), or thermoplastic polyolefin (TPO).

According to another aspect of the subject matter described herein, the conductive nanomaterials comprise silver nanowires (AgNWs), copper nanowires (CuNWs), nickel nanowires (NiNWs), gold nanowires (AuNWs), carbon nanotubes, graphene, or poly(3,4-ethylenedioxythiophene) polystyrene sulfonate (PEDOT:PSS).

According to another aspect of the subject matter described herein, the thin film epidermal electronic device is gas permeable.

According to another aspect of the subject matter described herein, the thin film epidermal electronic device is configured to be attached to human skin, and the one or more holes are configured to allow sweat to evaporate from the human skin.

According to another aspect of the subject matter described herein, each of the one or more holes has a diameter of between, and including, about 1 μm and 100 μm.

According to another aspect of the subject matter described herein, at least one of the one or more holes has a diameter of between, and including about 30 μm and 50 μm.

According to another aspect of the subject matter described herein, between, and including, about 30% and 50% of a surface area of the polymer layer is covered by the one or more holes.

According to another aspect of the subject matter described herein, the conductive nanomaterials are embedded on both a top surface and a bottom surface of the polymer layer.

According to another aspect of the subject matter described herein, conductive nanomaterials are also embedded on an inner surface of each of the one or more holes thereby connecting the conductive nanomaterials on the top surface and the conductive nanomaterials on the bottom surface.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The subject matter described herein will now be explained with reference to the accompanying drawings of which:

FIG. 1a is a schematic illustration of the fabrication process for porous HP-AgNW/TPU film. Optical (top) and SEM (bottom) images of: (FIG. 1b1 and FIG. 1b2) porous TPU film; (FIG. 1c1, FIG. 1c2) porous AgNW/TPU film; (FIG. 1d1, d2) porous HP-AgNW/TPU film. FIG. 1e is an optical image of porous TPU film. FIG. 1f is a graph illustrating pore size distribution. Scale bars: 40 μm in optical images, 20 μm in SEM images, and 5 μm in the insert images in FIG. 1c2 and FIG. 1d2;

FIG. 2a is a photograph of the porous HP-AgNW/TPU film showing good optical transmittance. The dotted lines show the boundary of the film. Scale bars, 40 mm. FIG. 2b is a graph illustrating sheet resistance as a function of number of dip-coating cycles. FIG. 2c is a graph illustrating transmittance spectrum of the porous TPU, AgNW/TPU and HP-AgNW/TPU films. FIG. 2d is a graph illustrating water vapor transmission of the porous TPU, AgNW/TPU and HP-AgNW/TPU films as a function of time. FIG. 2E is a graph illustrating relative resistance change when the porous AgNW/TPU and HP-AgNW/TPU films were immersed in saline solution. FIG. 2F is a photograph of the porous AgNW/TPU and HP-AgNW/TPU films after the peeling test on tape;

FIG. 3a illustrates a top view of an electrophysiological electrode on skin (top left). Scale bar, 5 mm. The image shown the EP electrode on skin when stretched (top right), compressed (bottom right) and twisted (bottom left). FIG. 3B is a graph illustrating relative resistance change as a function of tensile strain for the porous HP-AgNW/TPU film. 7 cycles of stretching and releasing were applied at each strain level. FIGS. 3c and 3d are graphs illustrating relative resistance change when the porous HP-AgNW/TPU film was subjected to bending (FIG. 3c) and cyclic bending (FIG. 3d), respectively;

FIGS. 4a-4f illustrate an application of the electrode in electrophysiological sensing. FIG. 4a is an optical image of the electrode transferred on the artificial skin. Scale bars, 400 μm in optical image. FIG. 4b is a photograph of the porous HP-AgNW/TPU electrodes attached onto left and right forearms for ECG testing. FIG. 4c is a photograph of three porous HP-AgNW/TPU electrodes attached onto the forearm for EMG testing. Scale bars, 10 mm in digital photograph FIG. 4d is a graph of electrode-skin impedance of commercial gel electrodes, AgNW/PDMS electrodes, porous AgNW/TPU electrodes, and porous HP-AgNW/TPU electrodes. FIG. 4e is a graph of ECG signals measured using the commercial gel electrodes and porous HP-AgNW/TPU electrodes placed nearby. FIG. 4f is a graph of EMG signals measured using the commercial gel electrodes and porous HP-AgNW/TPU electrodes placed nearby. The grip strength was measured by a hand dynamometer;

FIGS. 5a-5d illustrates an application of the electrode in wireless human-computer interface. FIG. 5a is a schematic diagram of the touch sensor based on the self-capacitance mode. The C_{parasitic} is the basic capacitance of the touch sensor system. FIG. 5b is a graph of capacitive touch values from the capacitive breakout. FIG. 5c is a schematic diagram illustrating the layout of the wireless human-computer interface with the porous HP-AgNW/TPU patterns as touch sensors. The insert on the bottom left shows the sleeve worn on the arm. FIG. 5d is a diagram illustrating an application of the touch sensor system in a video game, which in the illustrated example is Tetris. The four keys correspond to four different functions: moving left, moving down, rotating, and moving right, respectively;

FIG. 6 is a flow chart detailing some of the steps of a method according to some embodiments of the present disclosure;

FIGS. 7a1-7b2 include optical images of porous TPU films fabricated with different solution concentrations: (FIG. 7a1, FIG. 7a2) 1 wt % TPU+0.1 wt % PEG; (FIG. 7b1, FIG. 7b2) 2 wt % TPU+0.2 wt % PEG. Images at the bottom are the magnified view of the corresponding top images;

FIGS. 8a-8c include cross-section SEM images of (FIG. 8a) porous TPU film, (FIG. 8b) porous AgNW/TPU film, (FIG. 8c) porous HP-AgNW/TPU film;

FIG. 9 illustrates the stress-strain curves of porous TPU, porous AgNW/TPU and porous HP-AgNW/TPU film;

FIG. 10 includes photographs of the electrodes on skin before and after peeling off the electrodes after wearing for 7 days, showing no skin irritation;

FIG. 11 includes photographs of the AgNW/TPU and HP-AgNW/TPU electrodes on skin before and after peeling off;

FIG. 12a is a schematic of the LED circuit to demonstrate the double-side conductivity of the HP-AgNW/TPU film; and FIG. 12b is a photograph of the LED circuit. Copper wires are connected to different sides of the film through liquid metal;

FIG. 13a is a graph illustrating relative resistance changes during 1000 cycles of stretching and releasing with 10% tensile strain. For each cycle, the strain was hold for 10 s at zero and 10% strain, respectively. FIG. 13b is a graph illustrating relative resistances change in the time range of 8000-8100 s;

FIG. 14 includes a chart illustrating the relative resistance change as a function of tensile strain for the porous HP-AgNW/TPU film with larger strain;

FIG. 15 includes an optical image of the patterned electrode transferred on a wrinkled artificial skin, showing conformal contact between the electrode and the artificial skin;

FIG. 16 includes charts illustrating ECG signals measured using the commercial gel electrodes and porous HP-AgNW/TPU electrodes placed nearby, when the wrist was under continuous motion including stretching, bending, compression or vibration; and

FIG. 17 is a graph illustrating the capacitive touch value changes versus time.

### DETAILED DESCRIPTION

The present subject matter details methods and devices for stretchable conductive film prepared by embedding conductive nanowires, for example and without limitation, silver nanowires (AgNWs), at or just below the surface of a porous thermoplastic polyurethane (TPU) film fabricated by breath figure method. In addition to silver nanowires, those having ordinary skill in the art will appreciate that other nanomaterials can be used as well. For example and without limitation, instead of silver nanowires, copper nanowires (CuNWs), nickel nanowires (NiNWs), gold nanowires (AuNWs), carbon nanotubes, graphene, or poly(3,4-ethylenedioxythiophene) polystyrene sulfonate (PEDOT:PSS) can be used. Similarly, those having ordinary skill in the art will appreciate that instead of a TPU film, other polymers can be used in some embodiments of the present subject matter. It is particularly useful to have polymers that are insoluble in water, but soluble in an organic solvent such as THF. For example and without limitation, in addition to TPU, polystyrene-polybutadiene-polystyrene (SBS), or thermoplastic polyolefin (TPO) can also be used. The present disclosure uses silver nanowires and TPU as the primary substances for describing the subject matter of the present application, but those having ordinary skill in the art will appreciate that these materials can be replaced by any of the appropriate alternatives described and listed above.

The breath figure method is a simple, efficient and scalable self-assembly process to fabricate porous polymer films,^{21, 22} with no need for complex steps such as photolithography, vacuum evaporation, and etching. Compared with other methods, such as stamping or imprinting, this method has the advantage of low-cost, one-step, and template-free fabrication. Moreover, the porous film has good gas permeability for sweat evaporation to prevent skin irritation. The ultra-thin nature of this film enables a conformal contact with the skin, which can improve the signal quality of the electrophysiological (EP) sensing as well as the long-term wearing comfort. Utility of the thin-film electrodes for epidermal electronics is demonstrated in skin-mountable electrodes for EP sensing and textile-integrated touch sensors for human-machine interfaces.

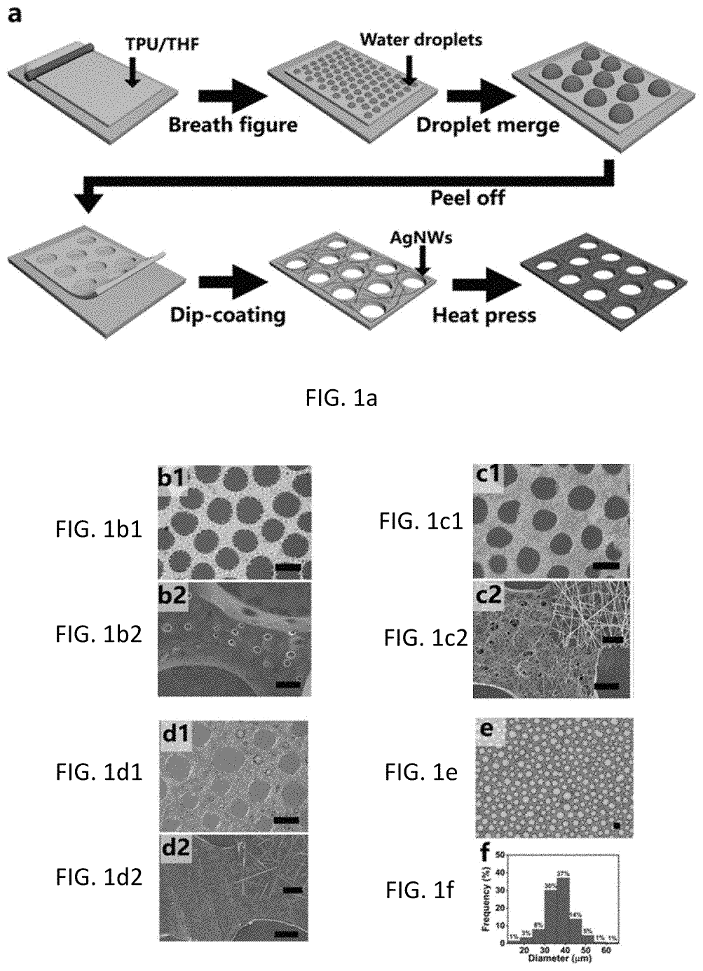

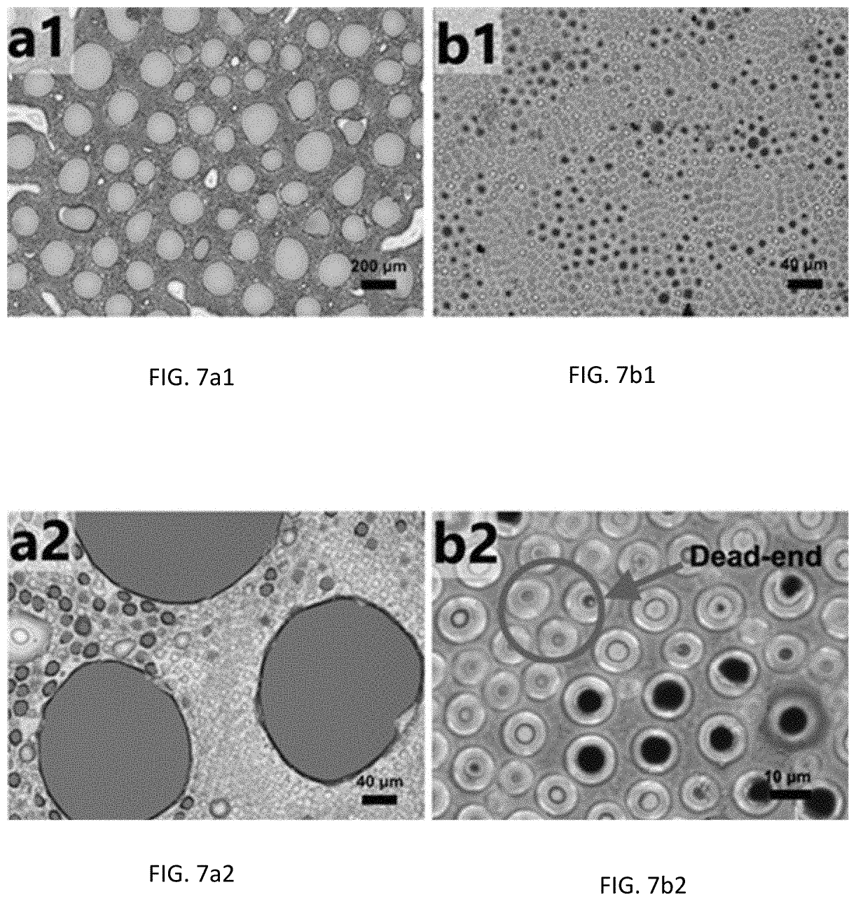

A schematic illustration of the fabrication process is shown in FIG. 1a. Briefly, a porous TPU film was prepared using a breath figure method, and AgNWs were dip-coated onto the surface. Heat press treatment was performed to melt the TPU surface and embed AgNWs just below its surface. In the breath figure process, tetrahydrofuran (THF) was used as the solvent. In addition, a small amount of polyethylene glycol (PEG) (TPU:PEG=10:1 in weight) was added to the solution to facilitate the ordered assembly of water droplets.²³ Evaporation of the organic solvent cooled down the substrate. The moisture condensed on the substrate and self-assembled into regular water drop arrays. Typically the convergence of water droplets should be avoided, which otherwise could lead to disordered structures.^{21, 22} However, in the present disclosure, the convergence of droplets contributed to the formation of through-pore structures. As shown in FIG. 1b1, FIG. 1b2 and FIG. 7, the pore size can be controlled by changing the concentration of the solution. Higher concentration (2 wt % TPU+0.2 wt % PEG) led to smaller pore size and more regular pore structure, but higher percentage of dead-end pores. On the other hand, when the concentration was too low (1 wt % TPU+0.1 wt % PEG), the resulting structure tended to be more irregular with pore diameter larger than 100 μm. Such large pores can become visible to naked eyes and pose a limit to the patterning resolution of the electrodes. The optimum solution concentration was found to be 1.5 wt % TPU and 0.15 wt % PEG. As can be seen from the optical and scanning electron microscope (SEM) images in FIG. 1b1, FIG. 1b2, and FIG. 1e, the porous structure was evenly distributed. The shape of the pores was close to circular with a pore diameter of ˜40 μm, and the pore coverage ratio is about 39% (FIG. 1e, FIG. 1f).

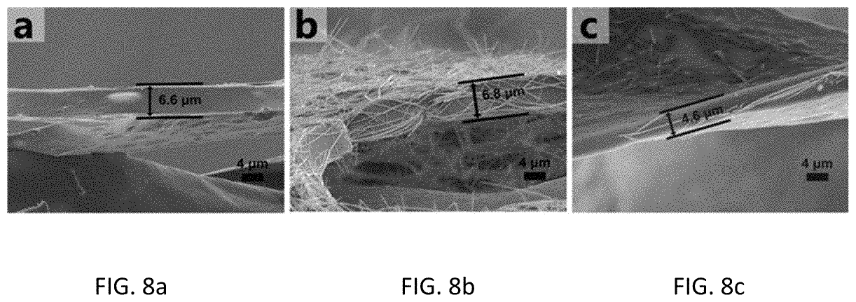

AgNWs were loaded onto the porous TPU film by dipping the film into the AgNW/water solution. The pore size of the TPU film was much larger than the length of AgNWs (˜20 μm). FIGS. 1c1 and 1c2 show the optical and SEM images of the porous AgNW/TPU film, where AgNWs were uniformly coated on the surface of TPU without hanging across pores. Dip-coated AgNWs on the TPU surface were easy to detach from the film, so a heat press treatment was introduced to enhance the adhesion.²⁴ Since the melting point of the TPU is around 130° C., a temperature of 150° C. was used for heat pressing. It can be observed from FIG. 1d1 and FIG. 1d2 that, after the heat press, majority of the AgNWs were inlaid inside TPU with only a small portion exposed on the surface. This morphology was further illustrated in the cross-sectional SEM images (FIG. 8). In some embodiments, a thickness of the AgNW/TPU film can be between, and including, about 1 μm and 100 μm. In some embodiments, for example and without limitation, the AgNW/TPU film can have a thickness of between, and including, about 3 μm and 8 μm. After the heat press is applied, the thickness of the porous AgNW/TPU film can decrease. For example and without limitation, in a test running of the method described herein, the thickness of the AgNW/TPU film decreased from 6.8 μm to 4.6 μm after the heat press.

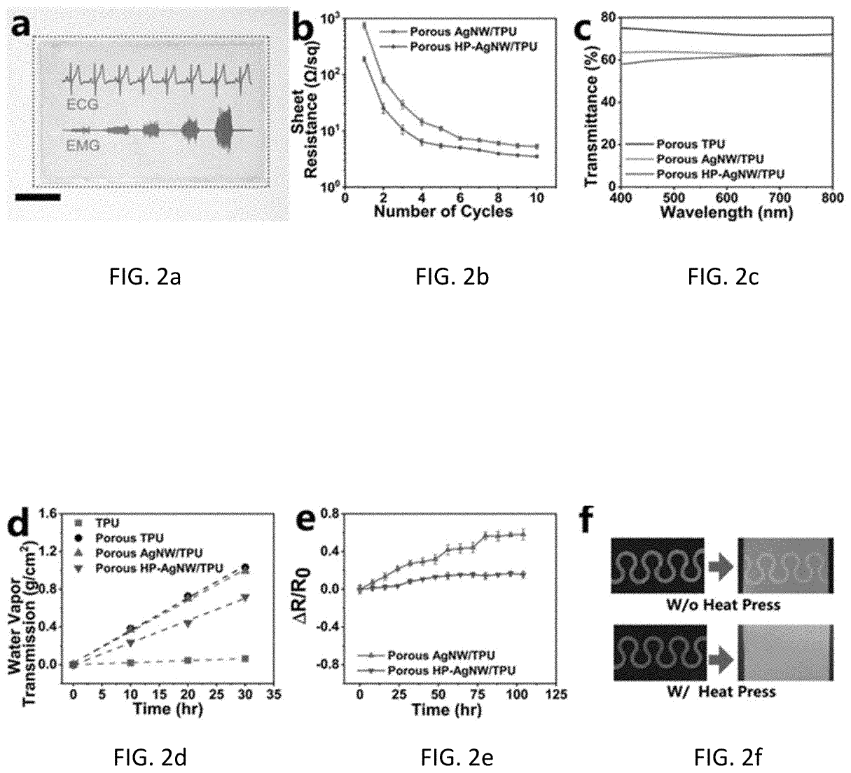

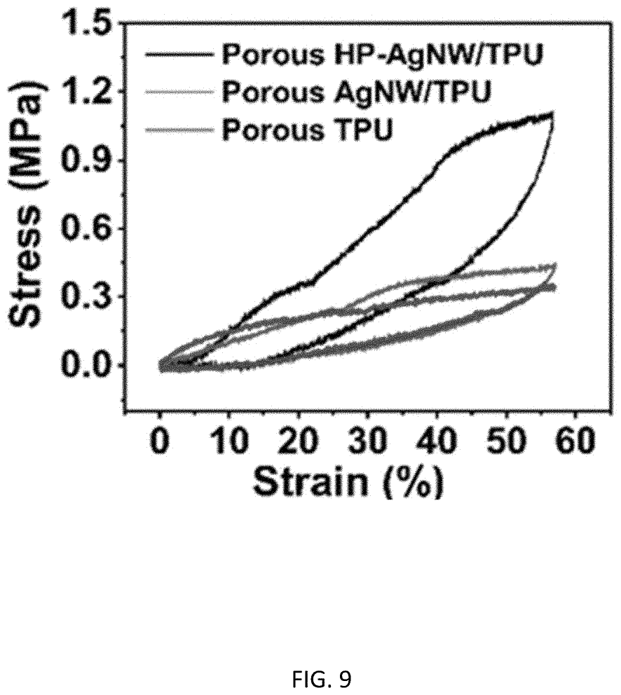

FIG. 2a shows the optical image of the porous HP-AgNW/TPU film. FIG. 2b shows the sheet resistance as a function of the number of dip-coating cycles. The sheet resistance decreased with coating cycles for the first 4 cycles. After that, the sheet resistance remained nearly constant with more coating cycles, reaching about 14.5 Ω/sq. Hence 4 cycles of dip coating were used in the fabrication. The heat press treatment further decreased the resistance, which can be attributed to the improved contact in the AgNW junctions caused by the pressure and thermal annealing during heat press.²⁵ For example, after heat press the sheet resistance of 4-cycle-coated film decreased to 7.3 Ω/sq. The porous structure of the film led to enhanced optical transparence compared to the solid film. The optical transmittance was 72% at 550 nm for the porous TPU film (FIG. 2c) and decreased to 63% after dip-coating AgNWs for 4 cycles. The transmittance further decreased to 61% after the heat press, due to the slightly increased width of the TPU skeleton and thus reduced pore coverage ratio. To investigate the mechanical properties of these porous films, the stress-strain curves of porous TPU, AgNW/TPU, and HP-AgNW/TPU films were tested. As shown in FIG. 9, the HP-AgNW/TPU film shows the highest stiffness, which is likely because the film became denser and the AgNWs formed stronger bonds with TPU and between themselves.

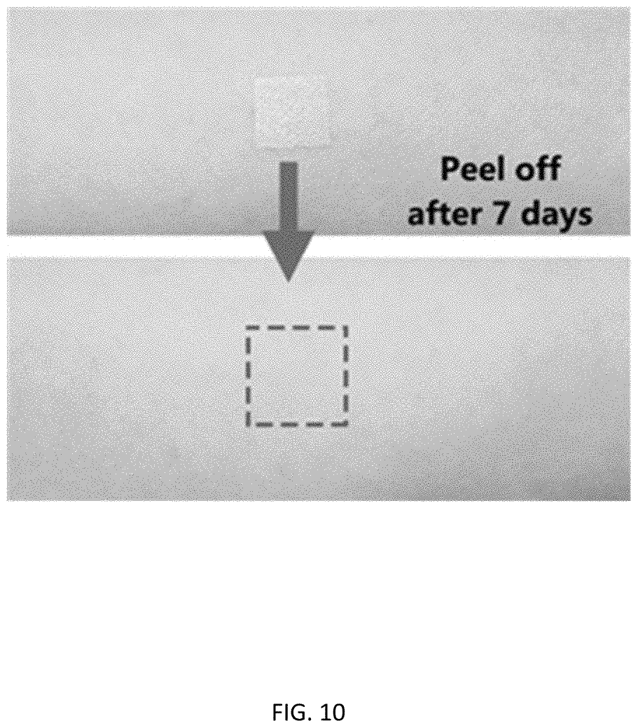

The water vapor transmission was evaluated based on the ASTM's E96 standard.²⁶ The porous TPU film exhibited dramatically enhanced vapor permeability compared to a TPU film without the porous structure (FIG. 2d). The water vapor transmission rates of solid TPU, porous TPU, porous AgNW/TPU, and porous HP-AgNW/TPU were measured to be 2, 38, 36, and 23 mg cm-2 h-1, respectively. Enhanced vapor permeability was hypothesized to render better long-term wearability. To test the hypothesis, a long-term on-skin wearability test was carried out (FIG. 10). After 7 days of wearing on human skin, no allergic reactions and sweat accumulation occurred. No difference was observed between the skin area that was covered by the film and the surrounding area. It is apparent that the through-pore structure allows sweat and moisture to escape through the film, reduces the likelihood of skin irritation, and improves the comfortableness and long-term wearability for wearable applications.

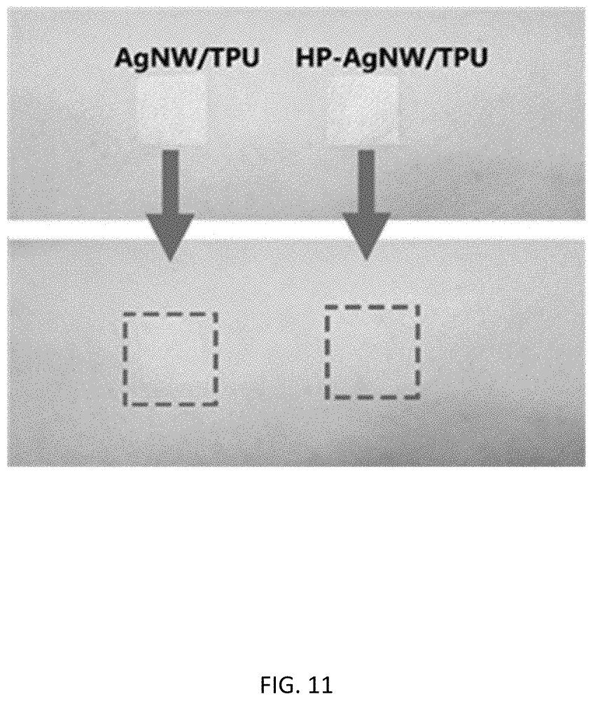

The films were immersed in a saline solution to demonstrate the long-term stability in sweat (FIG. 2e). After 100 hours, the resistance of porous AgNW/TPU and HP-AgNW/TPU films increased by 60% and 15%, respectively. Peel tests were performed between the film and a tape (FIG. 2f) and between the film and skin (FIG. 11). FIG. 2f shows that, the AgNW/TPU film can be easily peeled off by the tape (the image on the right shows the AgNWs transferred onto the tape), while the HP-AgNW/TPU film is much more stable. In addition, the AgNW/TPU film lost the conductivity after the peeling test, while the HP-AgNW/TPU film maintained the conductivity. As shown in FIG. 11, after peeling the AgNW/TPU film off the skin, some AgNWs were left on the skin. For the case of HP-Ag NW/TPU film, no obvious residue of AgNWs was observed on the skin. These experiments revealed that the heat press treatment can effectively improve the conductivity and stability for long-term wearable applications. By embedding AgNWs below the surface of TPU film, the resulting porous HP-AgNW/TPU film showed significantly enhanced adhesion between AgNWs and TPU and hence stability, with acceptable sacrifice of optical transmittance and vapor permeability.

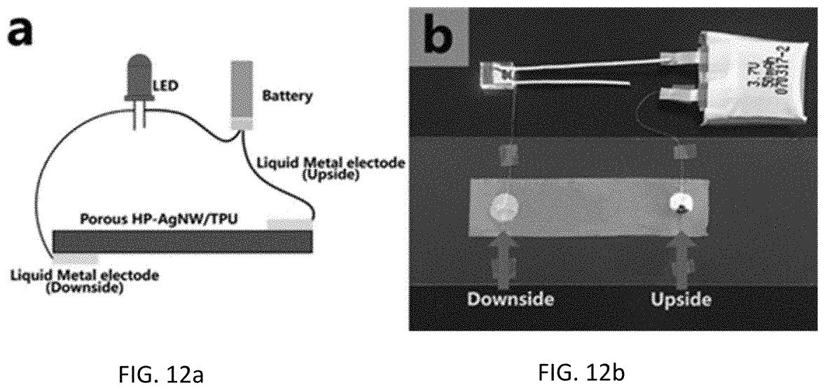

It is worth noting that the HP-AgNW/TPU film is not only conductive on the surface but also in the thickness direction. The top and bottom sides of the film were both electrically conductive; both sides were also connected by AgNWs on the edge of the pores through the thickness. It works like a bulk conductive material but does not require a large amount of conductive fillers, which could cause degradation in the mechanical properties. To demonstrate this property, the film was connected to an LED circuit and used as a double-sided conductor (FIG. 12). Two droplets of liquid metal were applied on the two sides of the film to interconnect with the LED. The lighted LED indicated that both sides of the film are electrically conductive and connected.

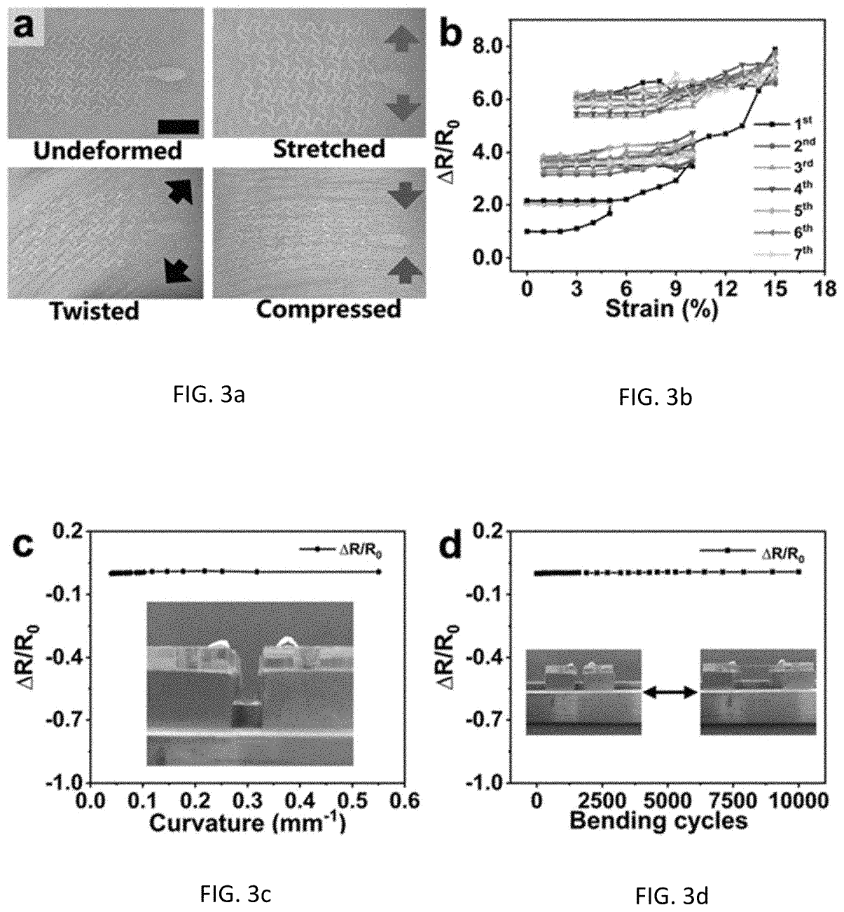

The porous HP-AgNW/TPU film can be laser-cut into different patterns. FIG. 3a shows a film electrode patterned into a filamentary serpentine structure with linewidth of 0.5 mm. The electrode can be used for electrocardiography (ECG) and electromyography (EMG) (to be discussed later). The ultrathin nature of the film enabled intimate contact with the skin. By spraying a small amount of breathable liquid bandage (Nexcare™, 3M) onto the skin, the film electrode can be easily laminated onto the skin. The film can be stretched, compressed, and twisted on skin (FIG. 3a) and completely recovered. After use, the film can be easily removed using a scotch tape and re-used for many times. The efficient lamination and removal demonstrated excellent user-friendliness of the porous HP-AgNW/TPU electrode.

FIG. 3b presents the change in resistance as a function of tensile strain. When 5% strain was applied to the porous HP-AgNW/TPU film, the resistance was doubled. When the strain was released to zero, the resistance had a slight drop of 10%. In the subsequent stretch-release cycles within the 5% strain, the resistance remained nearly constant and reversible. When stretched to 10% and 15% strains, the resistances increased about 4 and 7 times, respectively, compared to the initial value. But within the 10% and 15% strains, the similar trend to the 5% strain was seen: the resistance increased rapidly during the first stretch and then became reversible in the following stretch-release cycles with small resistance change. At each strain level, the film can be “programmed” by the first stretch, after which the resistance would change reversibly within the range defined by the first stretch.

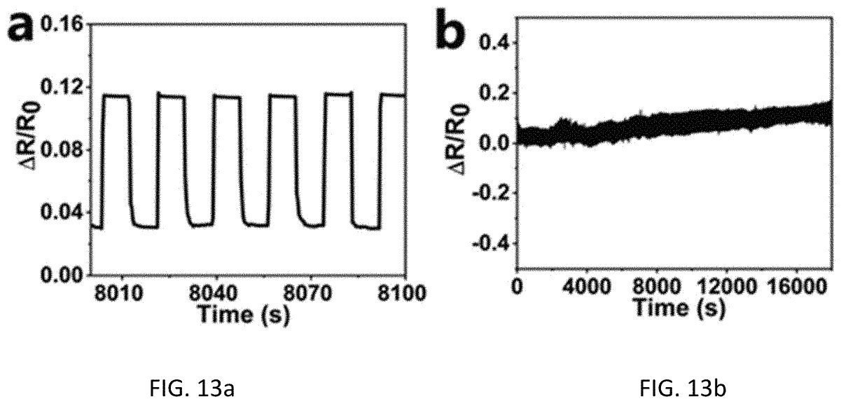

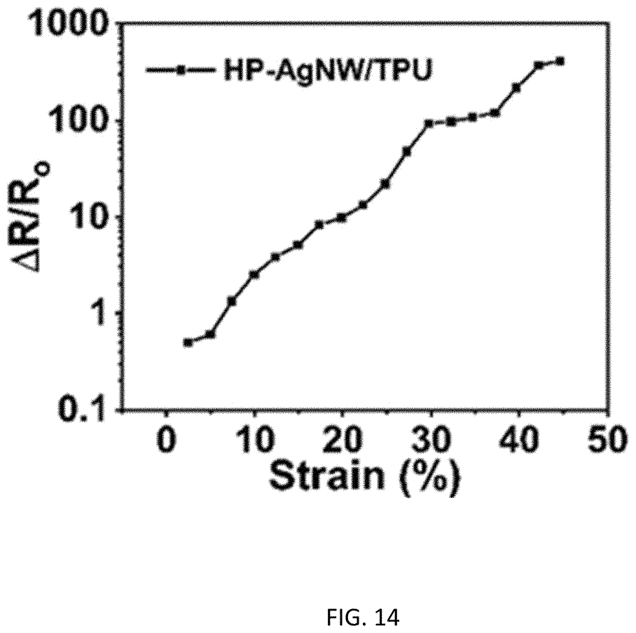

This “programmed” phenomenon is attributed to plastic deformation and buckling of the HP-AgNW/TPU film. During the first stretching, sliding between AgNWs and the porous TPU film occurs, which results in the surface buckling upon release of the strain. When the applied strain is relatively small (e.g., 5%), the surface buckling is the microstructural origin of the reversible change in the resistance.²⁷⁻³¹ Under larger applied strain (e.g., 10% or 15%), TPU undergoes plastic deformation, leading to irreversible structural deformation. For example, as shown in FIG. 3b, the permanent (plastic) strain was ˜3% when unloaded from the 15% strain. Nevertheless, between 3% and 15% strains the resistance was totally reversible, due to the surface buckling mechanism. The measurement of resistance changes for 1,000 cycles of stretching (10%) and release is shown in FIG. 13. After 1,000 cycles, the resistance increased less than 7%. In addition, the HP-AgNW/TPU film exhibited excellent flexibility. When the film was bent to a curvature of 0.55 mm⁻¹, the resistance only increased by 0.8% (FIG. 3c). After 10,000 cycles of bending, the resistance increased by 0.7% (FIG. 3d). Besides, the resistance change of the porous HP-AgNW/TPU film under large strain is shown in FIG. 14. It can be seen that the film remains conductive until 45% strain. The breakage strain of the HP-AgNW/TPU film is about 350%.

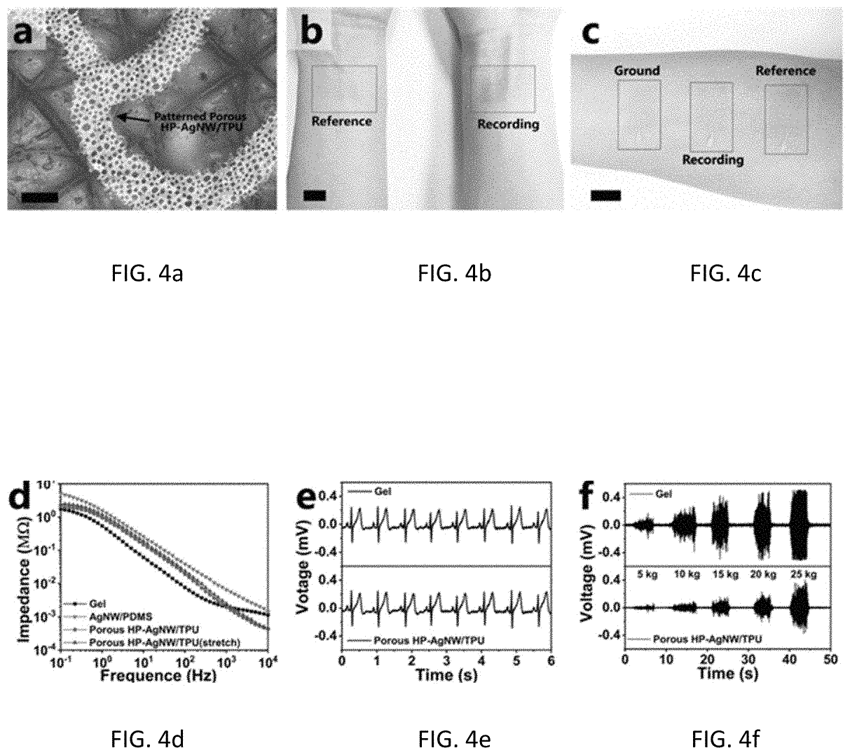

The porous HP-AgNW/TPU film is well suited for continuous monitoring of electrophysiological signals. ECG is commonly used to diagnose abnormal heart rhythms, while EMG can be used to analyze stimulation levels, muscle neuropathy, and motor behavior. In ECG and EMG measurements, conformal contact and low skin-electrode impedance is crucial for obtaining high signal-to-noise ratio.^{13, 17} To assess the contact between the porous HP-AgNW/TPU electrodes and the skin, artificial skin made of Exoflex was fabricated, which replicates the human skin with a similar Young's modulus. FIG. 4a and FIG. show the optical images of the electrode after transferred onto the artificial skin. It can be seen that the ultrathin electrode formed a conformal contact with the skin. FIG. 3a also shows conformal contact between the electrode and the skin.³²

The impedances for the original and stretched porous HP-AgNW/TPU electrodes were only slightly higher than that of the commercial Ag/AgCl gel electrodes (FIG. 4d), and lower than that of the solid AgNW/PDMS film (thickness 0.2 mm), which was demonstrated previously.³² The difference is related to the quality of the skin-electrode contact. The reduced thickness and the porous structure of the porous HP-AgNW/TPU film decrease the bending stiffness, leading to more conformal contact than the solid AgNW/PDMS film.

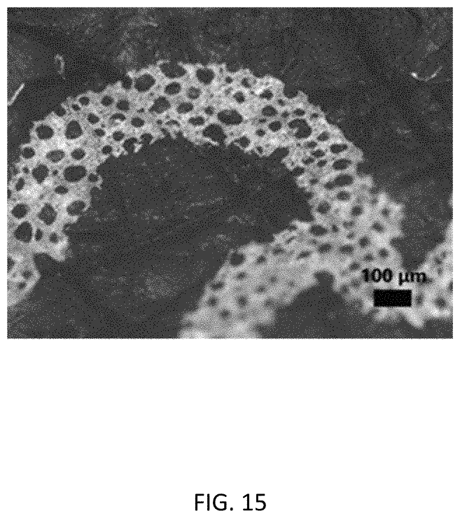

ECG and EMG signals obtained using the porous HP-AgNW/TPU electrodes were compared against those using the commercial Ag/AgCl gel electrodes, as shown in FIG. 4e and FIG. 4f. FIG. 4b and FIG. 4c show the placement of the electrodes for ECG and EMG testing, respectively. For ECG, the porous HP-AgNW/TPU electrodes provided comparable signal quality to the gel electrodes. The measured signal to noise ratio (SNR) of the ECG by the porous HP-AgNW/TPU electrodes was 7.0 dB, comparable to that measured by the gel electrodes, 7.1 dB. Under continuous wrist movement, motion artifacts were introduced to the signals measured using both the dry porous HP-AgNW/TPU electrodes and commercial gel electrodes (FIG. 15). The SNR of the ECG measured by HP-AgNW/TPU electrode and gel electrodes under movement are 6.3 dB and 6.2 dB, respectively. The HP-AgNW/TPU electrodes showed similar motion artifacts when compared with gel electrodes. The HP-AgNW/TPU electrodes showed higher susceptibility to motion artifacts compared with gel electrodes. For EMG, the signals corresponding to the muscle contraction for different grip strengths can be clearly distinguished. Notably the signal of the porous HP-AgNW/TPU electrodes was weaker than that of the gel electrodes, which is due to the different positions of the two types of electrodes (placed simultaneously). The measured SNR of the EMG by the reported electrodes (24.9 dB) was comparable to that measured by the gel electrodes (25.9 dB). Compared to commercial Ag/AgCl gel electrodes, the porous HP-AgNW/TPU electrodes do not require the conductive gel, which mitigates the skin irritation caused by the gel and the signal degradation due to dehydration of the gel.³ In view of the gas permeability demonstrated earlier, these experiments further illustrate the feasibility of the porous HP-AgNW/TPU electrodes for long-term, continuous health monitoring.

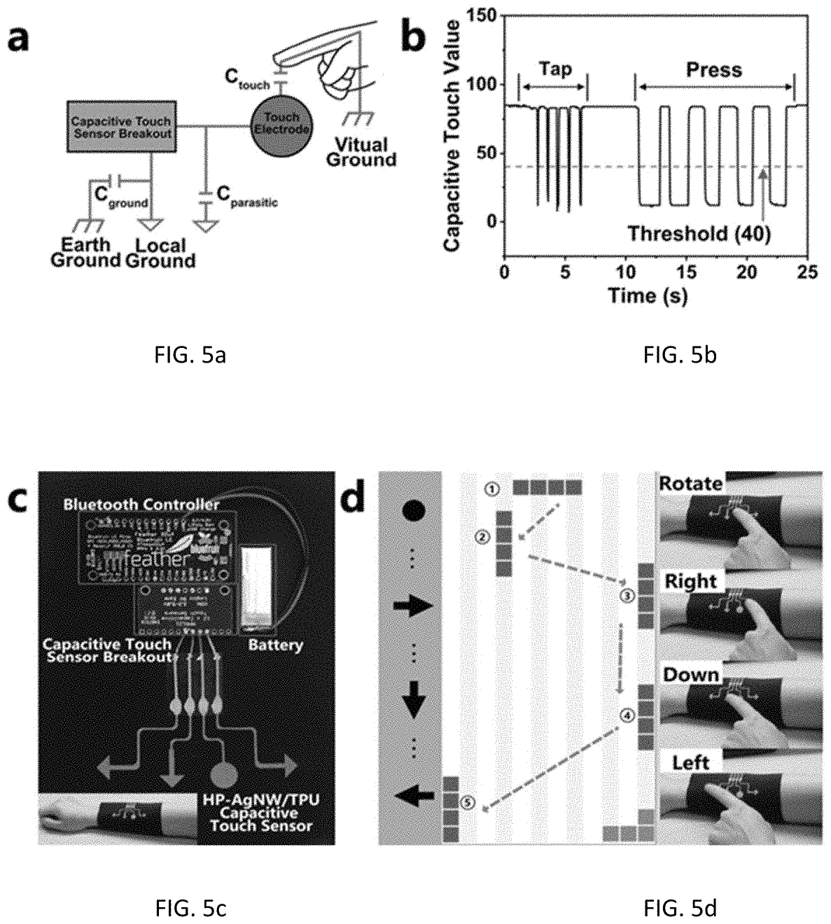

In addition to skin-mountable applications, the electrodes can be integrated with textiles. The present subject matter demonstrates the application of wireless capacitive touch sensing for human-machine interfaces. FIG. 5a shows the diagram of a touch sensor based on the self-capacitance mode.³³ Self-capacitive touch sensors use a single electrode to measure the apparent capacitance between the electrode and the virtual ground. Upon touched, the human-body capacitance is added to the self-capacitance circuit. As a result, the “touch” state can be determined by measuring the capacitance change. The capacitive breakout (MPR121 board) normalizes the capacitance into a “capacitance touch value”,³⁴ which can be compared with a threshold value (e.g., 40, as defined in this experiment) to identify the touch state. For example, if the value is lower than the threshold value, the electrode is being touched. FIG. 5b shows the capacitance touch value from the capacitive breakout. It is seen that finger “touch” (i.e., tap and press) can be clearly identified.

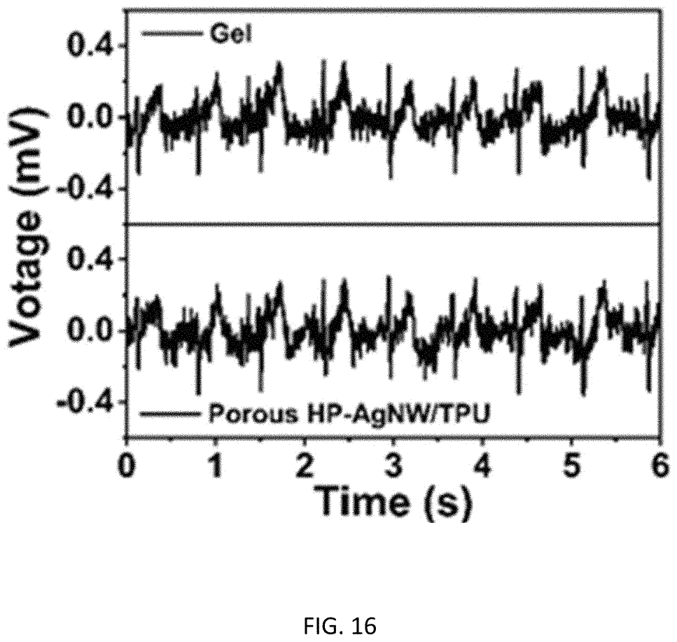

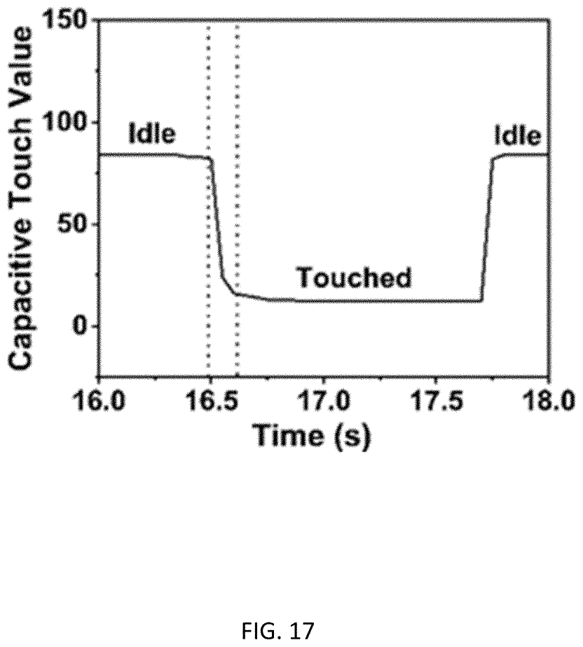

In addition, the sensitivity of the touch sensor system is defined as change rate of the reading value when a touch occurs,

$$Sensitivity = \frac{{V}_{n} - {V}_{t}}{{V}_{n}} \times 100 \% ,$$

where Vₙ is the non-trigger reading value and Vₜ is the trigger reading value. In our system, the sensitivity is calculated to be about 86%. The stability is defined as the dispersion of the touch sensor reading, which is about 1.65. The SNR of the system is about 35:1, and the response time is less than 0.1 s (FIG. 16). FIG. 17 illustrates how the capacitive touch value changes with time. These system parameters attest that our capacitance touch sensor system performs well for detecting finger touch.³⁵

To assemble a wireless touch sensor system (FIG. 5c), a piece of 50 mm×100 mm HP-AgNW/TPU film was integrated onto a fabric sleeve and patterned into four touch sensors/keys using laser cutting.²⁴ Each key was connected to a port of the touch sensor breakout, and the touch sensor breakout was connected to the Bluetooth controller (Feather 32u4) and battery to complete the assembly of the system. These four keys were assigned with four functions: left, down, rotation, and right. Each function can be activated when the finger touches the corresponding key.

Then the sleeve was put on the arm and used as a wireless human-machine interface to play Tetris on a computer display. The system was set as a Bluetooth keyboard in the computer. FIG. 5d shows the function of different keys and the movement trace of the tiles controlled by touching the keys. Upon pressing the keys of rotation, left, right and down on the sleeve, the tiles in the game produced corresponding real-time responses without noticeable delay. As illustrated in the video, the touch sensor was highly sensitive and accurate.

### Conclusion

Some embodiments of the present disclosure include facile fabrication of gas permeable, ultrathin, stretchable, semi-transparent and laser patternable electrodes for epidermal wearable applications, either directly mounted on skin or integrated with textiles, i.e., in garments configured to be worn by human users. The porous structure was enabled by a self-assembly process using the breath figure method, and the electric conductance was made possible by dip-coating AgNWs on the skeleton of the porous structure. The pore morphology and ratio of through-pores can be controlled by the concentration of solution in the breath figure process. With an optimal concentration of 1.5 wt % TPU+0.15 wt % PEG, through pores with size of around 40 μm was achieved. After heat press post-treatment, AgNWs were embedded below the surface of TPU, which improved the adhesion between AgNWs and TPU and conductivity of the film, while maintaining good transmittance and water vapor permeability. Vapor permeability is critical for the evaporation of sweat, which can effectively prevent adverse effects on the skin. The resistance of the film can be programmed by the first stretch and stabilized in the subsequent stretch-release cycles. The film was conductive on the top and bottom surfaces as well as through thickness. With a thickness of only a few micrometers and the porous structure, the electrode enabled a conformal contact with skin, leading to low electrode-skin impedance and excellent ECG and EMG signals on par with commercial gel electrodes. In the capacitive sensing application, the electrode was capable of responding to finger touches in real time with high accuracy. With unusual combination of the features including ultra-low thickness, semi-transparency, stretchability, gas permeability, facile fabrication, and laser patterning capability, the reported electrode offers a way for long-term wearable health monitoring and human-machine interfaces.

Methods

TPU film was purchased from Perfectex Plus LLC (Hot Melt Adhesive film model number #HM67-PA). PEG (average molecule weight 200) and THF were purchased from Fisher Scientific Reagent and used without further purification. AgNWs were synthesized using the polyol method.^{36, 37} Deionized water was purified using a Milli-Q system (MilliporeSigma).

Fabrication of Porous TPU Film on Glass Substrate

The porous film was fabricated by the breath figure method.^{22, 38} 1.5 g TPU and 0.15 ml PEG were dissolved into 100 ml THF solution. Then 5 ml of the solution was uniformly coated on a glass substrate (100 mm×200 mm) using a Meyer bar. After the bar coating process, the glass substrate was placed into a chamber with high humidity (relative humidity (RH): 99%, temperature: 25° C.) immediately. At this time, moisture was firstly condensed on the substrate and then merged to form a water droplet array. After the organic solvent and water droplets were evaporated, a porous TPU film was formed on the substrate after about 10 minutes. To facilitate the detachment of the porous TPU film from the glass substrate, a plastic frame was pressed onto the porous TPU film with the help of a double-sided adhesive tape. This way the porous TPU film can be peeled off the substrate. Afterwards, the porous TPU film was washed with DI water and dried at room temperature.

Coating AgNWs onto the Porous TPU Film

AgNWs were coated onto the film using dip coating. It is known that TPU swells in ethanol, methanol, isopropyl alcohol and other organic solvent, so AgNWs were dispersed in water in this experiment. Since TPU film is hydrophobic, plasma treatment for 3 minutes was introduced to render the surface hydrophilic. Then the treated TPU film was immersed into the AgNW solution (1 mg/ml) for 30 seconds and dried at 60° C. for 15 minutes. The coating and drying process was repeated several times to ensure sufficient AgNWs coated onto the porous TPU film.

Heat Press and Patterning the Porous AgNW/TPU Film into Designed Patterns

The AgNW/TPU film was placed in between two sheets of PTFE and subjected to heat press at 150° C. under a pressure of 3×105 Pa. After that, the heat pressed porous AgNW/TPU film (HP-AgNW/TPU) was fabricated. To pattern the porous HP-AgNW/TPU, the film was attached onto a PTFE film with the help of moisture generated by a humidifier. Then the film was cut into the designed pattern by a laser cutter (VLS6.60, Universal Laser Systems) with 0.1% power, 20% speed, and 1000 PPI. After removing the excessive material, porous HP-AgNW/TPU film with the designed pattern was fabricated.

Characterization

The microstructures of porous TPU, porous AgNW/TPU, and porous HP-AgNW/TPU films were characterized by scanning electron microscopy (SEM, FEI Quanta 3D FEG) and optical microscopy (ECLIPSE LV150N, Nikon). The transmittance of each film was measured using a UV-vis spectrophotometer (Cary 5000, Agilent). The resistance of each film was measured using a digital multimeter (34401A, Agilent). The water vapor transmission of each film was measured on a homemade test system based on ASTM E96 standard. The testing process is as follows: a 20 ml sized plastic bottle was filled with 15 ml distilled water, then sealed with a sample using a double-sided tape. The bottle was placed in a chamber with a temperature of 35° C. and RH of 40%±5%. The mass of the bottle was measured every 10 hours. The water vapor transmission was calculated based on the mass change. The electrode-skin impedance was obtained by performing a frequency sweep over a pair of electrodes placed at a distance of 7 cm on the forearm. The reported electrodes and gel electrodes were placed in adjacent. The electrode-skin impedance was measured using a potentiostat (Reference 600, Gamry Instruments). Electrocardiogram (ECG) and electromyography (EMG) signals were obtained using an amplifier (PowerLab 4/26, ADInstruments) with a sampling rate of 1 kHz. The SNR of ECG and EMG signals for both reported dry and commercial gel electrodes were calculated using the following equation,³²

$${SNR}_{dB} = 10 {{log}_{10} ( \frac{{A}_{signal}}{{A}_{noise}} )}^{2} = 20 {log}_{10} ( \frac{{A}_{signal}}{{A}_{noise}} ) ,$$

where A_{signal} is the root mean square of the biopotential signals (i.e., ECG or EMG signals in this study), Aₙₒᵢₛₑ is the root mean square of the noise collected in the settling trials. The Bluetooth controller (Feather 32u4 Bluefruit LE) and capacitive touch sensor breakout (MPR121) were purchased from Adafruit.

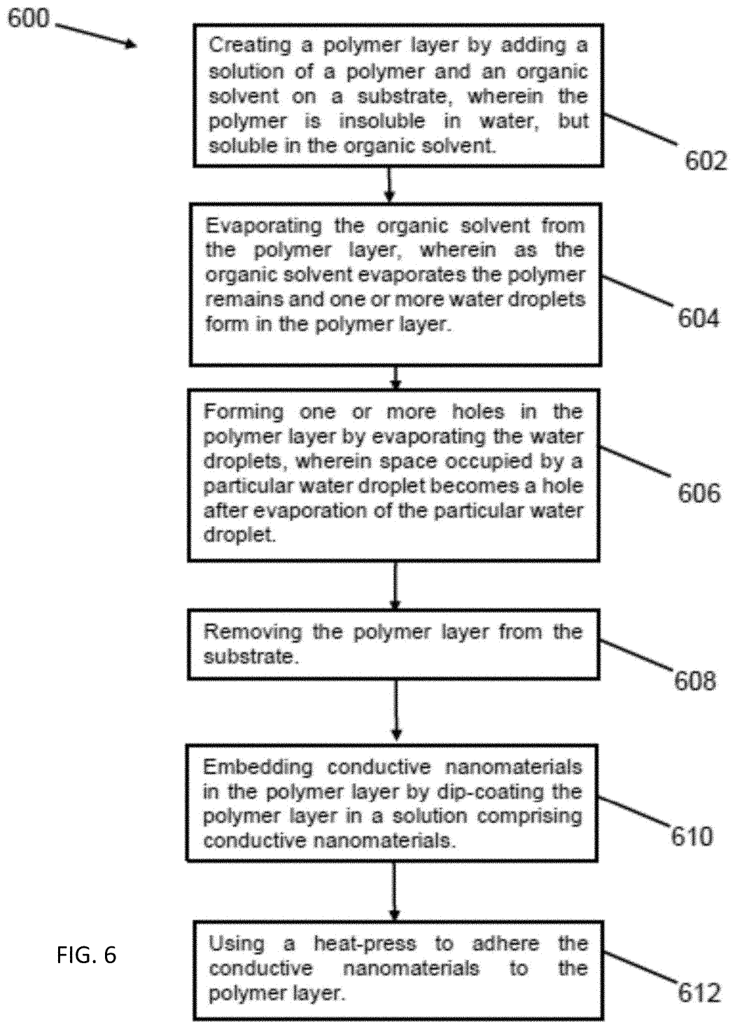

FIG. 6 illustrates a flow chart depicting steps in a method 600 according to some embodiments of the present disclosure. While the following description is presented in the form of an ordered list (i.e., first step, second step, etc.), those having ordinary skill in the art will appreciate that the steps in the method 600 can be performed in order or out of order. In fact, the steps of the method 600 can be performed in any suitable order. Furthermore, those having ordinary skill in the art will appreciate that additional steps may be performed during, between, before, or after any step listed in the following description, or in FIG. 6.

In some embodiments, a first step 602 of the method 600 comprises creating a polymer layer by adding a solution of a polymer and an organic solvent on a substrate, wherein the polymer is insoluble in water, but soluble in the organic solvent. In some embodiments, a second step 604 of the method 600 comprises evaporating the organic solvent from the polymer layer, wherein as the organic solvent evaporates the polymer remains and one or more water droplets form in the polymer layer. In some embodiments, a third step 606 of the method 600 comprises forming one or more holes in the polymer layer by evaporating the water droplets, wherein space occupied by a particular water droplet becomes a hole after evaporation of the particular water droplet. In some embodiments, a fourth step 608 of the method 600 comprises removing the polymer layer from the substrate. In some embodiments, a fifth step 610 of the method 600 comprises embedding conductive nanomaterials in the polymer layer by dip-coating the polymer layer in a solution comprising conductive nanomaterials. Finally, in some embodiments, a sixth step 612 of the method 600 comprises using a heat-press to adhere the conductive nanomaterials to the polymer layer.

The present subject matter can be embodied in other forms without departure from the spirit and essential characteristics thereof. The embodiments described therefore are to be considered in all respects as illustrative and not restrictive. Although the present subject matter has been described in terms of certain specific embodiments, other embodiments that are apparent to those of ordinary skill in the art are also within the scope of the present subject matter.

The disclosure of each of the following references is hereby incorporated herein by reference in its entirety.

### REFERENCES

1. Chen, D.; Pei, Q., Electronic Muscles and Skins: A Review of Soft Sensors and Actuators. Chem. Rev. 2017, 117, 11239-11268.
2. Liu, Y.; He, K.; Chen, G.; Leow, W. R.; Chen, X., Nature-Inspired Structural Materials for Flexible Electronic Devices. Chem. Rev. 2017, 117, 12893-12941.
3. Yao, S.; Swetha, P.; Zhu, Y., Nanomaterial-Enabled Wearable Sensors for Healthcare. Adv. Healthcare Mater. 2018, 7, 1700889.
4. Kim, D. H.; Ghaffari, R.; Lu, N.; Rogers, J. A., Flexible and Stretchable Electronics for Biointegrated Devices. Annu. Rev. Biomed. Eng. 2012, 14, 113-128.
5. Yao, S.; Ren, P.; Song, R.; Liu, Y.; Huang, Q.; Dong, J.; O'Connor, B. T.; Zhu, Y., Nanomaterial-Enabled Flexible and Stretchable Sensing Systems: Processing, Integration, and Applications. Adv. Mater. 2019, e1902343.
6. Kim, D. H.; Lu, N.; Ma, R.; Kim, Y. S.; Kim, R. H.; Wang, S.; Wu, J.; Won, S. M.; Tao, H.; Islam, A.; Yu, K. J.; Kim, T. I.; Chowdhury, R.; Ying, M.; Xu, L.; Li, M.; Chung, H. J.; Keum, H.; McCormick, M.; Liu, P., et al., Epidermal Electronics. Science 2011, 333, 838-843.
7. Trung, T. Q.; Lee, N. E., Flexible and Stretchable Physical Sensor Integrated Platforms for Wearable Human-Activity Monitoring and Personal Healthcare. Adv. Mater. 2016, 28, 4338-4372.
8. Kaltenbrunner, M.; Sekitani, T.; Reeder, J.; Yokota, T.; Kuribara, K.; Tokuhara, T.; Drack, M.; Schwodiauer, R.; Graz, I.; Bauer-Gogonea, S.; Bauer, S.; Someya, T., An Ultra-Lightweight Design for Imperceptible Plastic Electronics. Nature 2013, 499, 458-463.
9. Lipomi, D. J.; Vosgueritchian, M.; Tee, B. C.; Hellstrom, S. L.; Lee, J. A.; Fox, C. H.; Bao, Z., Skin-Like Pressure and Strain Sensors Based on Transparent Elastic Films of Carbon Nanotubes. Nat. Nanotechnol. 2011, 6, 788-792.
10. Yeo, W. H.; Kim, Y. S.; Lee, J.; Ameen, A.; Shi, L.; Li, M.; Wang, S.; Ma, R.; Jin, S. H.; Kang, Z.; Huang, Y.; Rogers, J. A., Multifunctional Epidermal Electronics Printed Directly Onto the Skin. Adv. Mater. 2013, 25, 2773-2778.
11. Gong, S.; Yap, L. W.; Zhu, B.; Cheng, W., Multiscale Soft-Hard Interface Design for Flexible Hybrid Electronics. Adv. Mater. 2019, 1902278.
12. Kim, H. W.; Kim, T. Y.; Park, H. K.; You, I.; Kwak, J.; Kim, J. C.; Hwang, H.; Kim, H. S.; Jeong, U., Hygroscopic Auxetic On-Skin Sensors for Easy-To-Handle Repeated Daily Use. ACS Appl. Mater. Interfaces 2018, 10, 40141-40148.
13. Yang, S.; Chen, Y. C.; Nicolini, L.; Pasupathy, P.; Sacks, J.; Su, B.; Yang, R.; Sanchez, D.; Chang, Y. F.; Wang, P.; Schnyer, D.; Neikirk, D.; Lu, N., “Cut-And-Paste” Manufacture of Multiparametric Epidermal Sensor Systems. Adv. Mater. 2015, 27, 6423-6430.
14. Kim, J. H.; Kim, S. R.; Kil, H. J.; Kim, Y. C.; Park, J. W., Highly Conformable, Transparent Electrodes for Epidermal Electronics. Nano Lett. 2018, 18, 4531-4540.
15. You, I.; Kim, B.; Park, J.; Koh, K.; Shin, S.; Jung, S.; Jeong, U., Stretchable E-Skin Apexcardiogram Sensor. Adv. Mater. 2016, 28, 6359-6364.
16. Gong, S.; Yap, L. W.; Zhu, B.; Zhai, Q.; Liu, Y.; Lyu, Q.; Wang, K.; Yang, M.; Ling, Y.; Lai, D. T. H.; Marzbanrad, F.; Cheng, W., Local Crack-Programmed Gold Nanowire Electronic Skin Tattoos for In-Plane Multisensor Integration. Adv. Mater. 2019, 31, e1903789.
17. Miyamoto, A.; Lee, S.; Cooray, N. F.; Lee, S.; Mori, M.; Matsuhisa, N.; Jin, H.; Yoda, L.; Yokota, T.; Itoh, A.; Sekino, M.; Kawasaki, H.; Ebihara, T.; Amagai, M.; Someya, T., Inflammation-Free, Gas-Permeable, Lightweight, Stretchable On-Skin Electronics with Nanomeshes. Nat. Nanotechnol. 2017, 12, 907-913.
18. Sun, B.; McCay, R. N.; Goswami, S.; Xu, Y.; Zhang, C.; Ling, Y.; Lin, J.; Yan, Z., Gas-Permeable, Multifunctional On-Skin Electronics Based on Laser-Induced Porous Graphene and Sugar-Templated Elastomer Sponges. Adv. Mater. 2018, 30, e1804327.
19. Fan, Y. J.; Li, X.; Kuang, S. Y.; Zhang, L.; Chen, Y. H.; Liu, L.; Zhang, K.; Ma, S. W.; Liang, F.; Wu, T.; Wang, Z. L.; Zhu, G., Highly Robust, Transparent, and Breathable Epidermal Electrode. ACS Nano 2018, 12, 9326-9332.
20. Cai, L.; Song, A. Y.; Wu, P.; Hsu, P. C.; Peng, Y.; Chen, J.; Liu, C.; Catrysse, P. B.; Liu, Y.; Yang, A.; Zhou, C.; Zhou, C.; Fan, S.; Cui, Y., Warming Up Human Body by Nanoporous Metallized Polyethylene Textile. Nat. Commun. 2017, 8, 496.
21. Bai, H.; Du, C.; Zhang, A.; Li, L., Breath Figure Arrays: Unconventional Fabrications, Functionalizations, and Applications. Angew. Chem. Int. Ed. Engl. 2013, 52, 12240-12255.
22. Zhang, A.; Bai, H.; Li, L., Breath Figure: A Nature-Inspired Preparation Method for Ordered Porous Films. Chem. Rev. 2015, 115, 9801-9868.
23. Ponnusamy, T.; Lawson, L. B.; Freytag, L. C.; Blake, D. A.; Ayyala, R. S.; John, V. T., In Vitro Degradation and Release Characteristics of Spin Coated Thin Films of PLGA with A “Breath Figure” Morphology. Biomatter 2012, 2, 77-86.
24. Yao, S.; Yang, J.; Poblete, F. R.; Hu, X.; Zhu, Y., Multifunctional Electronic Textiles Using Silver Nanowire Composites. ACS Appl. Mater. Interfaces 2019, 11, 31028-31037.
25. Cui, Z.; Poblete, F. R.; Zhu, Y., Tailoring the Temperature Coefficient of Resistance of Silver Nanowire Nanocomposites and Their Application as Stretchable Temperature Sensors. ACS Appl. Mater. Interfaces 2019, 11, 17836-17842.
26. Standard Test Methods for Water Vapor Transmission of Materials. ASTM International: West Conshohocken, Pa., 2016. 1-12.
27. Xu, F.; Wang, X.; Zhu, Y.; Zhu, Y., Wavy Ribbons of Carbon Nanotubes for Stretchable Conductors. Adv. Funct. Mater. 2012, 22, 1279-1283.
28. Zhu, Y.; Xu, F., Buckling of Aligned Carbon Nanotubes as Stretchable Conductors: A New Manufacturing Strategy. Adv. Mater. 2012, 24, 1073-1077.
29. Xu, F.; Zhu, Y., Highly Conductive and Stretchable Silver Nanowire Conductors. Adv. Mater. 2012, 24, 5117-5122.
30. Yao, S.; Zhu, Y., Nanomaterial-Enabled Stretchable Conductors: Strategies, Materials and Devices. Adv. Mater. 2015, 27, 1480-1511.
31. Jin, L.; Chortos, A.; Lian, F.; Pop, E.; Linder, C.; Bao, Z.; Cai, W., Microstructural Origin of Resistance-Strain Hysteresis in Carbon Nanotube Thin Film Conductors. Proc. Natl. Acad. Sci. 2018, 115, 1986-1991.
32. Myers, A. C.; Huang, H.; Zhu, Y., Wearable Silver Nanowire Dry Electrodes for Electrophysiological Sensing. RSC Adv. 2015, 5, 11627-11632.
33. Barrett, G.; Omote, R., Projected-Capacitive Touch Technology. Inf. Disp. 2010, 26, 16-21.
34. Nelson, C. Hello Capacitive Touch. https://learn.adafruit.com/circuit-playground-fruit-drums/hello-capacitive-touch (accessed Dec. 12, 2019).
35. Touch Sensor Application Note. https://github.com/espressif/esp-iot-solution/blob/master/documents/touch_pad_solution/touch_sensor_design_en.md (accessed March, 2020).
36. Sun, Y.; Xia, Y., Large-Scale Synthesis of Uniform Silver Nanowires Through A Soft, Self-Seeding, Polyol Process. Adv. Mater. 2002, 14, 833-837.
37. Zhu, Y.; Qin, Q.; Xu, F.; Fan, F.; Ding, Y.; Zhang, T.; Wiley, B. J.; Wang, Z. L., Size Effects on Elasticity, Yielding, and Fracture of Silver Nanowires: In Situ Experiments. Phys. Rev. B: Condens. Matter 2012, 85, 045443.
38. Wan, L. S.; Ke, B. B.; Zhang, J.; Xu, Z. K., Pore Shape of Honeycomb-Patterned Films: Modulation and Interfacial Behavior. J. Phys. Chem. B 2012, 116, 40-47.

What is claimed is:

1. A thin film epidermal electronic device comprising:
a polymer film having one or more holes therethrough;
wherein the polymer film comprises conductive nanomaterials embedded at or just below a surface of the polymer film;
wherein the conductive nanomaterials are connected to form a network of nanomaterials, thereby causing at least a part of the polymer film to act as an electrode;
wherein the polymer film is insoluble in water, but soluble in an organic solvent.

2. The thin film epidermal electronic device of claim 1, wherein the polymer film comprises thermoplastic polyurethane (TPU), polystyrene-polybutadiene-polystyrene (SBS), or thermoplastic polyolefin (TPO).

3. The thin film epidermal electronic device of claim 2, wherein the polymer film has a thickness of between, and including, about 1 μm and 100 μm.

4. The thin film epidermal electronic device of claim 1, wherein the conductive nanomaterials comprise silver nanowires (AgNWs), copper nanowires (CuNWs), nickel nanowires (NiNWs), gold nanowires (AuNWs), carbon nanotubes, graphene, or poly(3,4-ethylenedioxythiophene) polystyrene sulfonate (PEDOT:PSS).

5. The thin film epidermal electronic device of claim 1, wherein the thin film epidermal electronic device is gas permeable.

6. The thin film epidermal electronic device of claim 1, wherein the thin film epidermal electronic device is configured to be attached to human skin, wherein the one or more holes are configured to allow sweat to evaporate from the human skin.

7. The thin film epidermal electronic device of claim 1, wherein each of the one or more holes has a diameter of between, and including, about 1 μm and 100 μm.

8. The thin film epidermal electronic device of claim 1, wherein between, and including, about 30% and 50% of a surface area of the polymer film is covered by the one or more holes.

9. The thin film epidermal electronic device of claim 1, wherein the conductive nanomaterials are embedded on both a top surface and a bottom surface of the polymer film.

10. The thin film epidermal electronic device of claim 9, wherein conductive nanomaterials are also embedded on an inner surface of each of the one or more holes thereby connecting the conductive nanomaterials on the top surface and the conductive nanomaterials on the bottom surface.

11. A garment comprising:
a thin film epidermal electronic device, wherein the thin film epidermal electronic device includes a polymer film having one or more holes therethrough;
wherein the polymer film comprises conductive nanomaterials embedded at or just below a surface of the polymer film;
wherein the conductive nanomaterials are connected to form a network of nanomaterials, thereby causing at least a part of the polymer film to act as an electrode; and
wherein the polymer film is insoluble in water, but soluble in an organic solvent.

12. A method for making a thin film epidermal electronic device, the method comprising:
creating a polymer layer by adding a solution of a polymer and an organic solvent on a substrate, wherein the polymer is insoluble in water, but soluble in the organic solvent;
evaporating the organic solvent from the polymer layer, wherein as the organic solvent evaporates, the polymer remains and one or more water droplets form in the polymer layer;
forming one or more holes in the polymer layer by evaporating the water droplets, wherein space occupied by a particular water droplet becomes a hole after evaporation of the particular water droplet;
removing the polymer layer from the substrate;
embedding conductive nanomaterials in the polymer layer by dip-coating the polymer layer in a solution comprising conductive nanomaterials; and
using a heat-press to adhere the conductive nanomaterials to the polymer layer.

13. The method of claim 12, wherein the polymer comprises thermoplastic polyurethane (TPU) and the organic solvent comprises tetrahydrofuran (THF).

14. The method of claim 13 further comprising facilitating an ordered assembly of water droplets in the polymer layer by adding a quantity of polyethylene glycol (PEG) to the solution of TPU and THF;
wherein the PEG evaporates with the THF to leave a thin TPU film behind on the substrate.

15. The method of claim 12, wherein the polymer layer comprises thermoplastic polyurethane (TPU), polystyrene-polybutadiene-polystyrene (SBS), or thermoplastic polyolefin (TPO).

16. The method of claim 12, wherein the conductive nanomaterials comprise silver nanowires (AgNWs), copper nanowires (CuNWs), nickel nanowires (NiNWs), gold nanowires (AuNWs), carbon nanotubes, graphene, or poly(3,4-ethylenedioxythiophene) polystyrene sulfonate (PEDOT:PSS).

17. The method of claim 12, wherein the thin film epidermal electronic device is gas permeable.

18. The method of claim 12, wherein the thin film epidermal electronic device is configured to be attached to human skin, wherein the one or more holes are configured to allow sweat to evaporate from the human skin.

19. The method of claim 12, wherein the conductive nanomaterials are embedded on both a top surface and a bottom surface of the polymer layer.

20. The method of claim 19, wherein conductive nanomaterials are also embedded on an inner surface of each of the one or more holes thereby connecting the conductive nanomaterials on the top surface and the conductive nanomaterials on the bottom surface.
